(19) United States
(12) Patent Application Publication
(10) Pub. No.: US 2009/0117647 A1
(43) Pub. Date: May 7, 2009

(54) METHOD FOR ON-LINE OPTIMIZATION OF A FED-BATCH FERMENTATION UNIT TO MAXIMIZE THE PRODUCT YIELD

(75) Inventors: Babji Buddhi Srinivasa, Bangalore (IN); Jayant Modak Moreshwar, Bangalore (IN)

Correspondence Address: BUCHANAN, INGERSOLL & ROONEY PC, POST OFFICE BOX 1404, ALEXANDRIA, VA, 22313-1404, US

(73) Assignee: ABB Research Ltd., Zurich (CH)

(21) Appl. No.: 12/349,134

(22) Filed: Jan. 6, 2009

Related U.S. Application Data
(63) Continuation of application No. PCT/IB2006/001944, filed on Jul. 14, 2006.

(51) Int. Cl. C12M 1/02; G06F 19/00; G06F 17/10; G06G 7/66

(52) U.S. Cl. 435/289.1; 700/108; 703/2; 703/6

(57) ABSTRACT

A method for on-line optimization of fed-batch fermentation unit containing bacteria and nutrients is disclosed. Parameters of the fermenter model used in optimization calculations are estimated periodically to reduce the mismatch between the plant and the calculated values. The updated fermenter model is used to calculate the optimum sugar feed rate to maximize the product yield. The method/fermenter model is implemented as a software program in a PC that can be interfaced to plant control systems for on-line deployment in an actual plant environment. An on-line optimization system is useful to the plant operating personnel, to maximize the product yield from the fed-batch fermenter unit. In another aspect, a plant control system to control a fed-batch fermentation based on the described method is also disclosed.

### RELATED APPLICATION

This application claims priority as a continuation application under 35 U.S.C. §120 to PCT/IB2006/001944 filed as an International Application on 14 Jul. 2006 designating the U.S., the entire content of which is hereby incorporated by reference in its entirety.

### TECHNICAL, FIELD

The present disclosure deals with on-line optimization of a fed-batch fermentation unit. The fermentation unit is provided with computer based data acquisition and control system for the manipulation of the substrates feed rate profile in an optimum way to maximize the product yield from the fermenter.

### BACKGROUND INFORMATION

Fermentation processes are used widely in food and pharmaceutical industries to manufacture various products like alcohol, enzymes, antibiotics, vitamins etc. These processes involve a growth of microorganisms, utilizing the substrates and/or nutrients supplied and the formation of desired products. These processes are carried out in a stirred tank or other type of bioreactors with precise control of process conditions such as temperature, pH and dissolved oxygen. Due to complex biochemical reactions taking place within the cell, the control of substrates and/or nutrients at appropriate levels is essential for the formation of the products. Many fermentation processes are carried out in fed-batch mode wherein the substrates are fed continuously into the reactor over the fermentation period without withdrawing any fermentation broth. This type of feeding of the substrates has been found to overcome the effects such as substrate inhibition on the product yield. Usual industrial practice is to develop a reference profile for substrate feed rate based on operational experience and implement it in the plant with suitable adjustments to account for the actual conditions of the fermenter. This approach is empirical in nature and operator dependent, leading to variations in the product yield. Alternatively, a mathematical model of the fermentation process is used to calculate an optimum substrate flow rate profile off-line and implement it in the actual fermentation unit to maximize the product yield.

A number of different optimization methods and strategies for maximization of product yield of fed-batch processes were reported in literature. Optimization methods rely on a detailed mathematical model for computing an optimal feed profile and models considering both kinetics and transport phenomena occurring in the fermentation process have been used for optimization of fermentation units. The control variable used for maximizing product yield is generally the substrate (like sugar) feed rate at a constant substrate concentration.

Modak and Lim [1] formulated the feedback optimization of feed rate for fed-batch fermentation processes based on singular control theory and tested it on simplified fermenter models. Since fermentation processes exhibit time varying behavior, the success of a feedback control scheme depends on the reliability of the parameters of the model and uncertainties in the parameters leads to deterioration in the performance of the optimization scheme.

Cuthrell and Biegler, [9] proposed a simultaneous optimization and solution strategy based on SQP (Successive Quadratic Programming) and orthogonal collocation on finite elements and obtained results similar to that obtained with traditional method based on variational calculus for a simulated fed-batch fermenter model. The model considered did not include the effects of dissolved oxygen on biomass growth and product formation rates.

Kurtanjek [6] proposed a procedure based on orthogonal collocation technique and applied it for calculation of optimal feeding rate, substrate concentration in feed and temperature with constraints imposed on control and state variables. The fermenter model considered includes temperature effects on the specific growth rate constants.

Foss et al., [10] followed operator regime based modeling approach to express the fermenter model in a several local linear models and used SQP to optimize the average product formation rate. This approach requires considerable effort in formulation of the local linear models and data required for estimation of the parameters, of the order of few hundreds, is significantly much larger than what would be required for identification of a non-linear model. Hilaly et al., [11] demonstrated the real time optimization of a laboratory fed-batch fermenter unit through implementation of an optimal strategy derived from Pontryagin's Maximum principle. Improved yield and productivity compared to conventional fed-batch fermentation was reported. The fermenter model used in the optimization calculations was a simple one where the specific consumption rate of substrate and specific product formation rates were assumed to be linearly dependent on the specific growth rate of biomass and independent of concentration of dissolved oxygen in the broth. These assumptions are not valid in real plant environments. Van Impe and Bastin, [4] presented a methodology for optimal adaptive control and tested it on a simulated model of a fed-batch fermenter. However the method is applicable only for fermentation processes characterized by decoupling between biomass growth and product formation.

Banga et al, [7] used a stochastic direct search method to calculate the optimum feed rate for fed-batch fermentation processes and reported improved performance in simulation studies. However such open loop optimal control strategies will be inadequate in real situations due to the presence of disturbances and the time varying behavior of fermentation processes. In such situations the model parameters need to be updated on-line and the optimal trajectories need to be re calculated based on the updated model and state information.

Mahadevan et al, [12] presented an optimization scheme based on flatness and tested it by simulation on a simplified fed-batch fermenter model. Further work is needed to implement such optimization schemes on real fermenters as the model will be more complex than the one considered in their study.

Dhir at al, [2] dealt with the problem of maximizing cell mass and monoclonal antibody production from a fed-batch hybridoma cell culture in a lab scale bioreactor. They used a phenomenological model to represent the behavior of fermenter and used fuzzy logic based approach to update the model parameters to match the model predictions with plant data. An optimal control algorithm was formulated which calculated the process model mismatch at each sampling time, updated the model parameters and re-optimized the substrate concentrations dynamically throughout the course of the batch. Manipulated variables were feed rates of glucose and glutamine. Dynamic parameter adjustment was done using fuzzy logic techniques while a heuristic random optimizer optimized the feed rates. The parameters updated were specific growth rate and yield coefficient of lactate from glucose, chosen from sensitivity analysis. Studies carried out on a lab scale bioreactor showed substantial improvements in reactor productivity from dynamic re-optimization and parameter adjustment. Fuzzy logic based approach involves trial and error process that involves adjusting many parameters and is not very convenient for on-line deployment.

Iyer M S et al, [5] established a control scheme that includes off-line optimization, on-line model re-parameterization and on-line re-optimization of the recipe, for a fed-batch fermenter. It uses a rigorous phenomenological model whose parameters are adjusted using the one-step updating technique and a heuristic random optimizer for both off-line and on-line optimization. The objective function is to maximize the overall average rate of production of the desired product. While the model was adjusted every 5 hrs to keep it true to the process, on-line re-optimization was done once only every 4200 min (2 days and 22 hrs) because of slow process dynamics. The re-optimization was performed to determine the new batch time and feed rates starting from prevalent conditions at that time. Re-optimization was performed from any existing system state to determine the feed rates and remaining time of fermentation, such that the objective function was maximized. In the simulation studies carried out, an improvement of 10-14% in the productivity was obtained with on-line optimization when compared to off-line optimization.

Soni and Parker [3] developed an open loop optimal control policy in order to maximize the product concentration at the end of the batch with the substrate feed rate as the manipulated variable. A nominal controller based on shrinking horizon Quadratic Dynamic Matrix Control (SNQDMC) was implemented to track the reference trajectory determined from open loop optimization. Simulation studies showed good performance in tracking the reference trajectory and disturbance rejection while attaining the end of batch product concentration. SNQDMC algorithm is only a good approximation of using the non-linear fermenter model in optimization and is not tested on any experimental or real plants.

Bruemmer Bernd et. al. [15] have used a model of the fermenter to arrive at desired values for process parameters like partial pressure of oxygen, the conductivity and refractive index of the which are measured on-line. Any deviations from the desired values for these process variables are corrected by manipulating stirrer Rotations Per Minute (RPM)., air input, growth medium input and head pressure in the vessel. This approach is inadequate when mismatch occurs between the model and the actual plant due to some changes in the behavior of the fermentation process.

Though different approaches/algorithms were reported for optimizing the substrate feed rate in fed-batch fermentation processes, the methods do not address the requirements for on-line optimization of an industrial fed-batch fermentation unit. The optimization schemes often used simplified fermenter models and have not addressed the problem of the time varying nature of the model parameters adequately, particularly during deployment of the methods on-line in industrial environment. Some of the methods used heuristic random optimization techniques and approximate methods for model parameter estimation.

### SUMMARY

The best way to address all these issues is to use a model that adequately represents the phenomena occurring in the fermenter and use non-linear optimization techniques to estimate the model parameters and calculation of the optimal feed rate of the substrate to maximize the product yield. This scheme of parameter estimation and optimization is carried out periodically on-line based on the plant measurements and laboratory analysis results. This ensures that the model used in the optimization calculations is close to the behavior of the real fermentation unit.

Optimization of fed-batch fermentation units described above is an approximate method of reducing the model mismatch and optimizing substrate feeding profile. Factors such as variations in the quality of raw materials, characteristics of the initial charge media and disturbances in process conditions lead to mismatch between the model and the actual plant, adversely effecting the performance of the fermenter optimization system. The best way to address this issue is to use non-linear optimization techniques for updating the model on-line and optimization of the substrate feeding profile to maximize the product yield.

A method to calculate the optimum substrate feed rate is disclosed based on real time plant data and updated model to maximize the product yield from the fed-batch fermentation process. Since fermentation processes can be highly non-linear and vary temporally in their behavior, the model parameters and states can be updated on-line, to minimize the plant model mismatch. This approach can ensure that the model used in calculating the optimum feed rate is closer to the real plant behavior for better results of the optimization strategy. A non-linear optimization technique is used for both parameter estimation and optimization of the substrate feed rate. The on-line optimizer splits the future time horizon into stages and the optimal trajectory of the control variable is described piecewise as constant in each stage.

A method for on-line optimization of a fed-batch fermentation unit comprising: on-line measurement of plant parameters such as agitator speed, airflow rate, level measurement, sugar feed rate, percentage of carbon dioxide and oxygen in the vent gas and dissolved oxygen in the broth; storing of the on-line measurements/plant data as well as laboratory analysis results in a computer connected to the plant control system; fermenter model parameter re-estimation based on past and present plant data so as to reduce the mismatch between the plant data and the model calculation; on-line calculation of optimum sugar feed rate based on the current plant data and prediction of fermenter's future behavior so as to maximize the product yield.

In another aspect, a plant control system to control a fed-batch fermentation is disclosed. Such a plant control system comprises a fed-batch fermentation unit for fermentation of broth, the unit being subject to on-line measurement of plant parameters such as agitator speed, airflow rate, level measurement, sugar feed rate, percentage of carbon dioxide and oxygen in a vent gas and dissolved oxygen in the broth; and a computer connected to the plant control system capable of computing and storing the on-line measurements/plant data, as well as laboratory analysis results. At least the following calculations are computed: fermenter model parameter re-estimation based on past and present plant data so as to reduce the mismatch between the plant data and the model calculation, and on-line calculation of optimum sugar feed rate based on the current plant data and prediction of fermenter's future behavior so as to maximize the product yield.

### BRIEF DESCRIPTION OF THE DRAWINGS

FIG. 1 is a schematic representation of an exemplary fermentation unit.

FIG. 2 is schematic of an exemplary on-line optimization of fermenter unit.

### DETAILED DESCRIPTION

The on-line optimization method is comprised of the following steps:
Read the fermentation measurements from the control system and the laboratory analysis of the broth
Estimate current model parameters based on the measured as well as laboratory analysis data
Solve optimal control problem for future batch time horizon
Apply first stage value of the calculated optimal trajectory to the sugar feed flow controller

The calculation steps above are repeated every sampling period in a receding time horizon as the fermentation batch is in progress.

In the present approach, the improvement of about 5 to 10% in the product yield is expected as compared to the substrate feed rate strategy usually followed in the industrial fermenters.

In fed-batch fermentation operations, the substrate feeding profiles are adjusted to maintain the product yield of the batch. Due to a lack of appropriate tools, the substrate feeding profiles are adjusted based on heuristics and operational experience. Fed-batch fermenters are usually subject to changes in the initial conditions and disturbances in the process conditions leading to changes in the dynamic behavior with time, and the model parameters have to be adjusted to represent the process better. The present disclosure provides a novel method of updating the model parameters and uses the updated model for optimizing the substrate feed rate profile in a fed-batch fermentation unit. Based on the results of optimization calculations, changes to the substrate feed rate are implemented in the fermentation unit to maximize the yield. All the related mathematical calculations are implemented in a computer that is connected to the plant control system that provides the real time feedback of plant measurements like substrate feed flow rate, broth volume, air flow or agitator RPM, dissolved oxygen in the broth and percentage of oxygen and carbon dioxide in the vent gas of the fermentation unit.

The typical steps in implementation of the proposed on-line optimization strategy are as follows:
1. The process is started by charging the media into the fermentation vessel, starting the agitator and initiating the airflow through the broth.
2. All the plant operating parameters like air flow rate, agitator RPM, broth level, etc. are measured and stored in the control system and are available for the calculations.
3. Periodically, the broth samples are collected and analyzed in the lab for biomass yield in percentage by volume, concentration of sugar and product and the viscosity. The analysis results are stored in the plant computer control system.
4. With the initial conditions (broth volume, biomass concentration, product concentration, sugar concentration), the optimal sugar feed rate profile including the start time is calculated.
5. While the batch is in progress, following steps are executed:
   i. After completion of a predetermined schedule of fermentation startup, on-line estimation of fermenter model parameters is carried out based on the actual process data collected from the plant and laboratory analysis. The parameters are estimated by minimizing the error between the measured and predicted values for concentration of biomass, product, sugar, dissolved oxygen in the broth and composition (O2 and CO2) of vent gas. A non-linear optimization technique is used for minimizing the error between the predicted and measured values.
   ii. The new estimated parameters and the updated state variables (available from periodic laboratory analysis of concentrations of sugar, biomass and product and broth volume from the control system) are used in calculating the optimal sugar feed rate profile.
   iii. The calculated optimum sugar flow rate corresponding to the first stage of the future time horizon is assigned as set point to the sugar flow controller residing in the plant control system, which ensures that sugar flow rate is maintained at the optimum set-point.
   iv. Above sequence of steps (i to iii) are executed at every optimization calculation period.

This periodic re-estimation of the model parameters and updating the state variables while the batch is in progress is carried out as it helps in reducing the plant-model mismatch leading to improved performance of the optimizer.

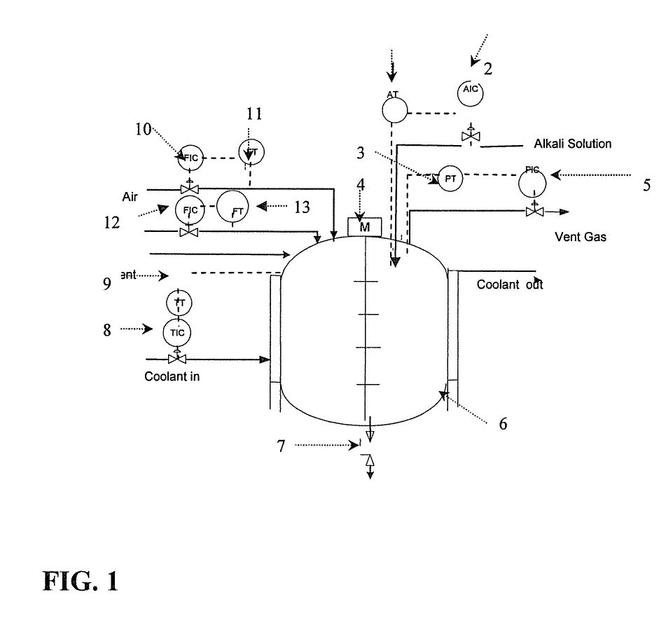

FIG. 1 illustrates a standard fermentation unit having the following automatic control schemes that are usually implemented in the fermenter unit control system:
pH control by manipulation of alkali flow rate
Fermenter temperature control by manipulation of coolant flow rate
Flow control for substrate addition
Pressure control by manipulation of vent gas valve
Flow control for inlet air
Adjustment of the agitator RPM through variable speed drive

The details of the various parts of the fermenter unit shown in FIG. 1 is as follows:

1—Fermenter broth pH transmitter.
2—Fermenter broth pH indicator controller.
3—Fermenter back pressure transmitter.

### 4—Agitator Motor.

5—Fermenter back pressure indicator controller.
6—Fermenter vessel.
7—Fermenter discharge valve.
8—Fermenter temperature indicator controller.
9—Fermenter temperature transmitter.
10—Air flow indicator controller.
11—Air flow transmitter.
12—Substrate flow transmitter.
13—Substrate flow indicator controller.

Various steps involved in the fermentation process are given below:
Biomass and the media from the lab pre seed vessel is charged into the main fermenter, which is provided with on-line sensors for measuring the pH, temperature, dissolved oxygen, volume of the broth, pressure of the vapor space and vent gas analysis for oxygen and carbon dioxide.
The pH controller automatically adjusts the flow rate of alkali solution to maintain the fermenter pH at a desired value.
After some time, sterile water is added to the fermenter to avoid dissolved oxygen (DO) starvation.
After the addition of sterile water, nutrient is added to provide the nutrients for cell growth.
Addition of substrate, like sugar solution is started when the concentration of sugar in the broth is lower than desired value and addition of sugar solution is continued till the end of the batch. In case the optimizer is enabled, the start time and flow rate of the substrate is determined by the on-line optimizer software.
During the course of the operation, one or two intermediate withdrawals of broth may be carried out for recovering the product.
The airflow is maintained at pre-defined flow set points.
The agitator RPM is maintained at two different levels: low speed initially and high speed for the remaining period of the batch.

Every few hours, broth sample is taken and analyzed in the laboratory for biomass yield in percentage by volume, concentration of sugar and alkali and the viscosity and product concentration.

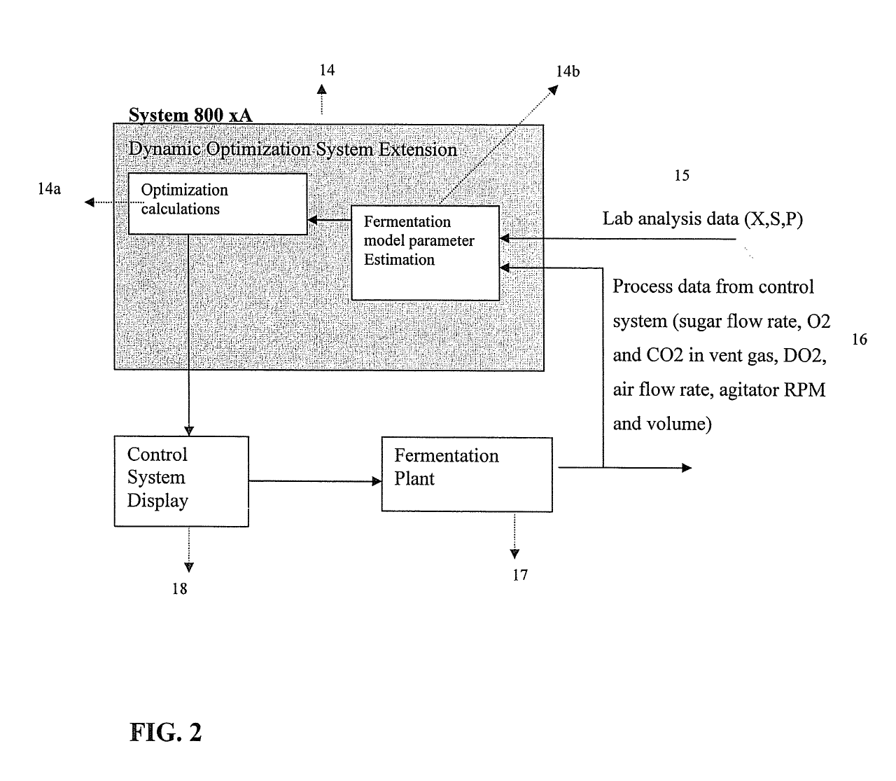

FIG. 2 is schematic of on-line optimization of fermenter unit. The optimization calculations are implemented as a software application in Dynamic Optimization System Extension (DOSE) of System 800×A, which is a standard process automation system developed by ABB based on the concept of object oriented approach to design and operation of process automation systems. DOSE is a software framework available in System 800xA and it provides a collection of tools for model-based application. The fermenter optimization method described above is implemented in DOSE as per the procedure described in the reference manual [13], which is incorporated by reference. DOSE provides the equation solvers and non-linear optimization routines required for simulation and model parameters estimation. Standard features of DOSE and System800xA are used for configuration, execution, display and storage of results obtained during simulation and parameter estimation of the fermenter model.

DOSE, shown in FIG. 2, parts 14, 14(a) and 14(b), can be interfaced with control systems and any other software systems supporting the Object linking and embedding for Process Control standard [hereby referred to as the OPC (Object linking and embedding for Process Control) standard] for data communications. This will help in implementing the fermenter model on-line with the provision of a data read/write facility with external systems. DOSE provides a collection of tools for model-based applications like simulation, parameter estimation and optimization, shown in FIG. 2, part 14(b). A spreadsheet plug-in provides the interface to configure the data required for carrying out the simulation, estimation or optimization and storing the calculation's results.

The schematic system for on-line optimization of fermenter unit to maximize the product yield is also discussed herein after.

### AN EXEMPLARY EMBODIMENT OF ON-LINE FERMENTER OPTIMIZATION SYSTEM IN CONTROL SYSTEM

In the present case, an unstructured [cell is represented by single quantity like cell density (g dry wt/L)] and unsegregated [view the entire cell population to consist of identical cells (with some average characteristics)] model approach is used for modeling the fermentation process, as this modeling approach is more amenable for on-line applications like estimation and optimization.

The following assumptions are made while developing the model:
Density of the fermentation broth is assumed to be same as that of water (1 gm/ml).
The cell growth is influenced by sugar and oxygen concentrations. The dependency on sugar and oxygen is modeled with Contois kinetics, which is an extension of Monod's kinetics [14].
The product formation rates are influenced by sugar and oxygen concentration, with sugar exerting inhibitory type control over the production rates.
The sugar consumption is accounted for by cell growth, product formation and maintenance
The oxygen mass transfer rates are influenced by agitation rate, air supply rate and viscosity.
Cell growth follows a sequence of lag period, growth period and maintenance or decay period and this is considered in the model.
Perfect mixing in the fermenter.
Temperature and pH in the fermenter are maintained at constant values and the model does not include the effect of these variables on the fermenter performance.

As described above, it has been found that the product yield from the fermenter can be maximized by periodically optimizing the sugar feeding profile. The parameters of the model used in the optimization calculations are updated on-line periodically based on actual plant measurements and laboratory analysis to account for the non-linear and time varying behavior of the batch fermentation process. The optimizer is depicted in FIG. 2, part 14(a). The parameters are obtained by minimizing the error between measured and predicted values of variables like concentration of product, sugar concentration, biomass, dissolved oxygen and O₂ and CO₂ concentration in the vent gas. A constrained non-linear optimization technique is used to minimize the error. Measured values of the concentration of biomass, product and sugar in the broth are available from lab analysis, shown in FIG. 2, part 15, every few hours and measurements of composition of vent gas and dissolved oxygen concentration are available from the control system every few minutes, shown in FIG. 2, part 16.

The fermenter model, shown in FIG. 2, part 14(b), along with the required equation solvers and optimization routines are implemented as a software application module using Dynamic Optimization System Extension framework available in System 800 ax. This is helpful in interfacing the fermenter model software with any other software system supporting the OPC standard for data transfer. The optimizer's output is displayed on a control system display, shown in FIG. 2, part 18, before being fed to the fermentation plant, shown in FIG. 2, part 17.

A brief description of the mathematical model of the Fermentation Unit is outlined below.

Fermentation processes are usually carried out as fed-batch operation in stirred tank type of bioreactors with precise control of process conditions such as temperature, pH and dissolved oxygen. These fermentation units are usually subjected to unmeasured disturbances leading to large variation in the product yields. Mathematical models can be used for a better understanding of the fermentation process and also to improve the operation to reduce the product variability and optimal utilization of the available resources.

The present disclosure deals with on-line optimization of fed-batch fermentation process to maximize the product yield. Fermentation processes are characterized by highly non-linear, time variant responses of the microorganisms and some of the model parameters are re-estimated on-line to minimize the modeling errors, such that the model used in optimization calculations is close to the real plant behavior. A constrained non-linear optimization technique is used to calculate the optimal sugar feed rate profiles for the fed-batch fermentation unit.

The optimization calculations are implemented in a computer that is interfaced with the microprocessor based system used for operation and control of the fermentation unit. Details of the fermenter model and the optimization strategy are given in the following section.

### Total Mass:

The fed-batch process operation causes a volume change in the fermenter. This is calculated by:

$$\frac{d}{d t} = ( V ) = {F}_{in} + {F}_{str} - {F}_{out} - {F}_{loss}$$

Where V is the volume of the fermenter broth, Fᵢₙ is the flow rate of sugar entering the fermenter, Fₒᵤₜ accounts for the spillages and Fₗₒₛₛ accounts for evaporation losses during fermentation. The sterile water and nutrient addition term is included as Fₛₜᵣ. Cell mass in the fermenter broth is determined by the following equation:

$$\frac{d}{d t} ( xV ) = {F}_{in} {x}_{in} - {F}_{out} x + {μ}_{D} xV - {K}_{dx} xV$$

where x is the concentration of biomass in the broth at any time, xᵢₙ is the concentration of biomass in sugar solution and specific growth rate μ_{D} is given by

$${μ}_{D} = {μ}_{max} \frac{S}{{K}_{s} X + S} \frac{{C}_{L}}{{K}_{O} X + {C}_{L}}$$

S and C_{L} are the concentration of sugar and dissolved oxygen in the broth.

### Product in Fermenter Broth:

The product formation is described by non-growth associated product formation kinetics. The hydrolysis of the product is also included in the rate expression

$$\frac{d}{d t} ( pV ) = {F}_{in} {p}_{in} - {F}_{out} p + {π}_{R} xV - {k}_{d} pV$$

where, P is the concentration of product in the broth at any time, Pᵢₙ is the concentration of product in sugar solution, π_{R} is the specific product formation rate defined as:

$${π}_{R} = {π}_{max} \frac{S}{{K}_{SP} + S + {K}_{i} {S}^{2}} \frac{{C}_{L}}{{K}_{OP} X + {C}_{L}}$$

### Sugar in Fermenter Broth:

The consumption of sugar is assumed to be caused by biomass growth and product formation with constant yields and maintenance requirements of the microorganism.

$$\frac{d}{d t} ( SV ) = {F}_{in} {S}_{F} - {σ}_{D} XV - {F}_{out} S$$

where S_{F} is the concentration of sugar in sugar solution and σ_{D} is the specific sugar consumption rate defined as:

$${σ}_{D} = \frac{{μ}_{D}}{{Y}_{X / D}} + \frac{{π}_{R}}{{Y}_{P / D}} + {m}_{D}$$

### Dissolved Oxygen in Fermenter Broth:

The consumption of oxygen is assumed to be caused by biomass growth and product formation with constant yields and maintenance requirements of the microorganism. The oxygen from the gas phase is continuously being transferred to the fermentation broth.

$$\frac{d}{d t} ( {C}_{L} V ) = {F}_{in} {C}_{L , in} - {F}_{out} {C}_{L} + {k}_{L} a ( {C}_{L}^{*} - {C}_{L} ) V - 1000 {σ}_{O} XV$$

where C_{L,in} and C_{L} are concentration of dissolved oxygen in the sugar solution entering and broth respectively. σₒ is the specific oxygen consumption rate, defined as:

$${σ}_{O} = \frac{{μ}_{D}}{{Y}_{X / O}} + \frac{{π}_{R}}{{Y}_{P / O}} + {m}_{O}$$

The overall mass transfer coefficient, k_{L}a is assumed to be function of agitation speed
(rpm), airflow rate (Fₐᵢᵣ), viscosity (μ) and fermentation broth volume and is defined as:

$${k}_{L} a = {( {k}_{L} a )}_{0} {( \frac{rpm}{{rpm}_{0}} )}^{a} {( \frac{{F}_{air}}{{F}_{air , 0}} )}^{b} {( \frac{{μ}_{0}}{μ} )}^{c} {( \frac{{V}_{0}}{V} )}^{d}$$

where the subscript 0, refers to nominal conditions. The saturation of dissolved oxygen concentration, C*_{L}, is related to the partial pressure of oxygen, p_{O2}, using Henry's law:

$${C}_{L}^{*} = \frac{{p}_{O 2}}{h}$$

$$DO 2 = ( {C}_{L} / {C}_{L}^{*} ) ⋆ 100$$

where DO2, is the measurement of dissolved oxygen available from the plant measurements

### Gas Phase Oxygen:

The gas phase is assumed to be well mixed, and the airflow rate is assumed to be constant.

$$\frac{d}{d t} = ( \frac{{V}_{g} {Py}_{O 2}}{RT} ) = \frac{{F}_{air} {P}_{0}}{{RT}_{0}} ( {y}_{O 2 , in} - {y}_{O 2} ) - \frac{{k}_{L} a}{1000 \times 32} ( {C}_{L}^{*} - {C}_{L} ) V$$

Where y_{O2,in} and y_{O2} are mole fraction of oxygen in the air and fermenter vent gas, P and T are the pressure and temperature of vapor space in the fermenter, P₀ and T₀ are pressure and temperature at normal conditions and R is the gas constant and V_{g} is the volume of vapor space in the fermenter.

### Gas Phase Carbon Dioxide:

The introduction of variables that are easy to measure while being important in their information content has been very helpful in predicting other important process variables. One such variable is CO₂ evolution, from which cell mass may be predicted with high accuracy. In this work, CO₂ evolution is assumed to be due to growth, product biosynthesis and maintenance requirement. The carbon dioxide evolution is given by:

$$\frac{d}{d t} = ( \frac{{V}_{g} {Py}_{CO 2}}{RT} ) = \frac{{F}_{air} {P}_{0}}{{RT}_{0}} ( {y}_{CO 2 , in} - {y}_{CO 2} ) + \frac{{σ}_{CO 2}}{44} XV$$

Where y_{CO2,in} and y_{CO} are mole fraction of carbon dioxide in the air and fermenter vent gas and σ_{CO2}, is the specific carbon dioxide evolution rate defined as:

σ_{CO2}=Y_{CO2/X}μ_{D}+Y_{CO2/P}π_{R}+m_{CO2}

### Optimization Strategy

The objective is to maximize the product yield at the end of the batch and the related objective function is defined as

$$J = {\int }_{t = {t}_{0}}^{{t}_{f}} ( p \cdot v ) \cdot d t$$

Above objective function is maximized with respect to sugar feed rate profile and subject to the fermenter model described above.

The optimal sugar feed rate is calculated subject to the following constraints:

0<Fᵢₙ<Fₘₐₓ

Vₘᵢₙ<V<Vₘₐₓ

δFₘᵢₙ<ΔFᵢₙ<δFₘₐₓ

### Where

t₀ initial batch time
t_{f} final batch time
Fᵢₙ feed rate of sugar/substrate calculated by the optimizer
Fₘₐₓ maximum allowed flow rate of sugar
Vₘᵢₙ minimum volume of the broth
Vₘₐₓ maximum value of the broth
δFₘᵢₙ minimum value of rate of change of Fᵢₙ
δFₘₐₓ maximum value rate of change of Fᵢₙ

A list of various kinetic parameters used in the model are listed below:

### Kinetic Parameters:

Growth
Maximum specific growth rate: μₘₐₓ (h⁻¹)
Contois saturation constant: K_{S}
Oxygen limitation constant for growth K_{O} (mg/L)
Cell decay rate constant: K_{dx} (h⁻¹)

Product Formation
Specific rate of production: Πₘₐₓ (g/L/h)
Contois constant: Kₛₚ (L⁻²/g⁻²)
Inhibition constant for product formation: Kᵢ (g/l)
Oxygen limitation constant for product: K_{OP} (mg/L)
Product hydrolysis rate constant: K_{d} (h⁻¹)

Sugar Consumption
Cellular yield constant: Y_{X/D} (g cellmass/g sugar)
Product yield constant: Y_{P/D} (g product/g sugar)
Maintenance coefficient on sugar: m_{D} (h⁻¹)

Oxygen Consumption
Cellular yield constant: Y_{X/O} (g cellmass/g oxygen)
Product yield constant: Y_{P/O} (g product/g oxygen)
Maintenance coefficient on oxygen: mₒ (h⁻¹)

Oxygen Transfer
Nominal mass transfer coefficient: k_{L}a₀ (h⁻¹)
Nominal rpm: rpm₀
Nominal air flow rate: F_{air,0} (m³/h)
Nominal viscosity: μ₀ (cP)
Nominal volume: V₀ (L)
Henry's constant: h
Constants: a, b, c, d

Gas phase oxygen
Normal pressure: P₀ (atm)
Gas phase volume: V_{g} (L)
Gas constant: R (atm m³ gmol⁻¹ K⁻¹)
Normal temperature: T₀ (K)

Gas Phase Carbon Dioxide
Cellular yield constant: Y_{CO2/X} (g carbon dioxide/g cell mass)
Product yield constant: Y_{CO2/P} (g carbon dioxide/g product)
Maintenance coefficient on oxygen: m_{CO2} (per h)

Initially, parameters of the fermenter model in DOSE are estimated with plant data in off-line mode and tuned to match with real plant data. The tuned model will be used to optimize the sugar feed rate to maximize the product yield of the fermenter.

In the on-line mode, the model will receive the real-time data like air flow rate, agitator RPM, sugar flow rate, dissolved oxygen and vent gas composition (oxygen and carbon dioxide) from the plant control system and also the analysis of fermentation broth (biomass yield in percentage volume, concentration of sugar, alkali and product) from the laboratory once every few hours. This combination of real-time process data and off-line laboratory data is used to estimate the model parameters. Periodic re-estimation of model parameters reduces the model mismatch and brings the model behavior closer to real operating conditions of the fermenter. The updated model will be used to calculate the optimum sugar feed rate profile. This cycle of parameter estimation, calculation of optimum sugar feed rate profile and implementation of the optimum sugar flow rate in the plant control system are repeated periodically in real-time.

It will be appreciated by those skilled in the art that the present invention can be embodied in other specific forms without departing from the spirit or essential characteristics thereof. The presently disclosed embodiments are therefore considered in all respects to be illustrative and not restricted. The scope of the invention is indicated by the appended claims rather than the foregoing description and all changes that come within the meaning and range and equivalence thereof are intended to be embraced therein.

### REFERENCES

1. Modak J M and H C Lim, “Feedback Optimization of fed-batch Fermentation” Biotechnology and Bio Engineering, 30, 528-540, 1987.
2. Dhir at al, “Dynamic Optimization of Hybridoma growth in a fed-batch Bioreactor”, Biotechnology and Bioengineering, 67(2), 197-205, 2000
3. Soni A S and R S Parker, “Closed loop control of fed-batch bioreactors: A shrinking approach”, Ind. Eng. Chem. Res., 43, 3381-3393, 2004
4. Van Impe J F and G Bastin, “Optimal adaptive control of fed-batch fermentation process”, Control Eng. Practice, 3, 939-954, 1995
5. Iyer M S et al, “Dynamic Reoptimization of a Fed-Batch Fermenter”, Biotechnology and Bioengineering, 63(1), 10-21, 1999.
6. Kurtanjek K, “Optimal nonsingular control of fed-batch fermentation”, Biotechnology and Bioengineering, 37, 814-823, 1991.
7. Banga J R et al, “Stochastic dynamic optimization of batch and semicontinuous bioprocesses”, Biotechnol. Prog., 13, 326-335, 1997
8. Chen C and C Hwang, “Optimal control computation for differential algebraic process systems with general constraints”, Chem. Eng. Comm., 97, 9-26, 1990
9. Cuthrell J E and L T Biegler, “Simultaneous optimization and solution methods for batch reactor control profiles”, Computers Chem. Eng., 13, 49-62, 1989.
10. Foss B A et al., “Non-linear predictive control using local models—applied to a batch fermentation process”, Control Eng. Practice, 3, 389-396, 1995
11. Hilaly A K et al., “A study on real time optimization of a fed-batch recombinant escherichia coli fermentation”, Control Eng. Practice, 3, 485-493, 1995.
12. Mahadevan et al, “Differential flatness based non-linear predictive control of fedbatch bioreactors”, Control Engineering Practice, 9, 889-899, 2001.
13. 1 KGC 003 952 Dynamic Optimization Reference Manual V.2.1.1, 2005
14. M L Schuler and F Kargi, “Biochemical Engineering Basic Concepts”, Prentice Hall, 2002.
15. Patent No. DE3927856, 1991-02-28. “Process control of cell culture fermentation and production.—uses oxygen partial pressure, conductivity and refractive index to control and optimise process relative to model predictions”.

What is claimed is:

1. A method for on-line optimization of a fed-batch fermentation unit comprising:
a. on-line measurement of plant parameters such as agitator speed, airflow rate, level measurement, sugar feed rate, percentage of carbon dioxide and oxygen in the vent gas and dissolved oxygen in the broth;
b. storing of the on-line measurements/plant data as well as laboratory analysis results in a computer connected to the plant control system;
c. fermenter model parameter re-estimation based on past and present plant data so as to reduce the mismatch between the plant data and the model calculation;
d. on-line calculation of optimum sugar feed rate based on the current plant data and prediction of fermenter's future behavior so as to maximize the product yield.

2. A method for on-line optimization of a fed-batch fermentation unit according to claim 1, wherein the model parameters are estimated by means of:
a. measuring the values of the concentration of biomass, product and sugar in the broth through lab analysis every few hours;
b. measuring the composition of vent gas and dissolved oxygen concentration from the control system every few minutes.

3. A method for on-line optimization of fermentation unit according to claim 1, wherein the on-line estimation of fermenter model parameters is initiated after completion of a pre-determined schedule of fermentation startup, with the actual process data collected during this startup phase being used to estimate the parameters, using a computer connected to the control system.

4. A method for on-line optimization of a fed-batch fermentation unit according to claim 1, wherein the model parameters are estimated by minimizing the error between the measured and predicted values for concentration of biomass, product, sugar, dissolved oxygen in the broth and composition (O2 and CO2) of vent gas using a non-linear optimization technique.

5. A method for on-line optimization of a fed-batch fermentation unit according to claim 1, wherein the optimal sugar feed rate is calculated using the current operating conditions (broth volume, product concentration, sugar concentration, dissolved oxygen) and future average profiles of airflow rate and agitator RPM and downloaded periodically as set-point for sugar feed flow controller in the control system.

6. A method for on-line optimization of a fed-batch fermentation unit according to claim 1, wherein the mathematical model of the fermenter predicts the future product yield and other operating parameters such as concentration of dissolved oxygen, biomass and product, percentage of carbon dioxide and oxygen in the vent gas.

7. A plant control system to control a fed-batch fermentation, comprising:
a fed-batch fermentation unit for fermentation of broth, the unit being subject to on-line measurement of plant parameters such as agitator speed, airflow rate, level measurement, sugar feed rate, percentage of carbon dioxide and oxygen in a vent gas and dissolved oxygen in the broth; and
a computer connected to the plant control system capable of computing and storing the on-line measurements/plant data, as well as laboratory analysis results, wherein at least the following calculations are computed:
fermenter model parameter re-estimation based on past and present plant data so as to reduce the mismatch between the plant data and the model calculation, and
on-line calculation of optimum sugar feed rate based on the current plant data and prediction of fermenter's future behavior so as to maximize the product yield.
